(19) United States
(12) Patent Application Publication
(10) Pub. No.: US 2015/0208967 A1
(43) Pub. Date: Jul. 30, 2015

(54) Biological Information Measurement Device And Pulse Oximeter

(71) Applicant: KONICA MINOLTA, INC., Chiyoda-ku (JP)

(72) Inventors: Norihiro Tateda, Sakai-shi (JP); Masaharu Kanazawa, Suita-shi (JP); Hitoshi Kamezawa, Kyoto-shi (JP)

(21) Appl. No.: 14/420,302

(22) PCT Filed: Jul. 18, 2013
(86) PCT No.: PCT/JP2013/069463

§ 371 Date: Feb. 6, 2015

(30) Foreign Application Priority Data
Aug. 9, 2012 (JP) 2012-176943

(51) Int. Cl. A61B 5/1455; A61B 5/00; A61B 5/024

(52) U.S. Cl. CPC A61B 5/14552; A61B 5/024; A61B 5/6826

(57) ABSTRACT

The biological information measurement device includes a body part and a wearable part that is fixed on the body part and is to hold a finger of a living body. The biological information measurement device includes a light source unit and a light receiving unit that are arranged so as to oppose each other with the finger therebetween when the finger is held by the wearable part. The body part includes a power supply unit and an electric circuit that includes a signal processing unit. The signal processing unit acquires a digital value concerning a pulse wave from a signal output from the light receiving unit by the light receiving unit receiving light emitted from the light source unit and passing through the finger. A first length of the body part in a longitudinal direction is greater than a second length of the wearable part in the longitudinal direction.

### TECHNICAL FIELD

The present invention relates to biological information measurement devices and pulse oximeters.

### BACKGROUND ART

A pulse oximeter that is capable of measuring oxygen saturation of blood (SpO₂) is known. In this pulse oximeter, a measurement part that is worn on a part of a body of a subject shines light onto the part of the body, and SpO₂ is derived based on the amount of light that passes through or is reflected off the part of the body.

As for this pulse oximeter, a device in which a light source, a sensor, a processor, an amplifier, and the like are arranged in an integrated housing has been proposed (e.g., Patent Document 1). This configuration reduces the manufacturing cost for the device, and makes the device less prone to breakage. Another device that includes a body part to be worn on a wrist and a probe to be fixed on a finger with a strip-shaped tape has been proposed (e.g., Patent Document 2).

### PRIOR ART DOCUMENT

### Patent Document

Patent Document 1: European Patent No. 1830695

Patent Document 2: Japanese Patent Application Laid-Open No. 2005-110816

### SUMMARY OF THE INVENTION

### Problems to be Solved by the Invention

With the technology disclosed in Patent Document 1 above, a robust integrated housing is worn on a finger. This makes it difficult to bend the finger at its joint, and thus wearing the device for a long time is a heavy burden for subjects. With the technology disclosed in Patent Document 2 above, work of winding a tape around a finger to fix the device on the finger is bothersome, and, due to the presence of the body part worn on a wrist and a cable extending from the body part to a probe fixed on the finger, long-time wearing of the device is a heavy burden for subjects.

The present invention has been conceived in view of the above-mentioned problems, and aims to provide a biological information measurement device and a pulse oximeter that are easily worn on a finger, and can reduce a burden imposed on subjects by long-time wearing on the finger.

### Means for Solving the Problems

In order to solve the above-mentioned problems, a biological information measurement device according to one aspect includes a body part and a wearable part that is fixed on the body part and is to hold a finger of a living body, and includes a light source unit and a light receiving unit that are arranged so as to oppose each other with the finger therebetween when the finger is held by the wearable part. In the biological information measurement device, the body part includes a power supply unit and an electric circuit that includes a signal processing unit. The signal processing unit acquires a digital value concerning a pulse wave from a signal output from the light receiving unit by the light receiving unit receiving light that is emitted from the light source unit and passes through the finger. In the biological information measurement device, a first length of the body part in a longitudinal direction of the body part is greater than a second length of the wearable part in the longitudinal direction.

A pulse oximeter according to another aspect includes a body part and a wearable part that is fixed on the body part and is to hold a finger of a living body, and includes a light source unit and a light receiving unit that are arranged so as to oppose each other with the finger therebetween when the finger is held by the wearable part. In the pulse oximeter, the body part includes a power supply unit and an electric circuit that includes a signal processing unit. The signal processing unit acquires a value concerning oxygen saturation of blood from a signal output from the light receiving unit by the light receiving unit receiving light that is emitted from the light source unit and passes through the finger. In the pulse oximeter, a first length of the body part in a longitudinal direction of the body part is greater than a second length of the wearable part in the longitudinal direction.

### Effects of the Invention

The biological information measurement device according to one aspect and the pulse oximeter according to another aspect are easily worn on a finger, and can reduce a burden imposed on subjects by long-time wearing on the finger.

### BRIEF DESCRIPTION OF DRAWINGS

FIG. 1 schematically shows the appearance of a biological information measurement device according to an embodiment.

FIG. 2 schematically shows the appearance of the biological information measurement device according to the embodiment.

FIG. 3 schematically shows the appearance of the biological information measurement device according to the embodiment.

FIG. 4 schematically shows the structure of the biological information measurement device according to the embodiment.

FIG. 5 shows a YZ cross section taken along an alternate long and short dash line V-V of FIG. 4.

FIG. 6 is a block diagram showing a functional structure of the biological information measurement device according to the embodiment.

FIG. 7 is a block diagram showing the functional structure of the biological information measurement device according to the embodiment.

FIG. 8 illustrates how the biological information measurement device according to the embodiment is worn on a finger.

FIG. 9 illustrates how the biological information measurement device according to the embodiment is worn on the finger.

FIG. 10 illustrates how the biological information measurement device according to the embodiment is worn on the finger.

FIG. 11 schematically shows the structure of a biological information measurement device according to a modification.

FIG. 12 schematically shows the structure of a biological information measurement device according to another modification.

FIG. 13 illustrates how the biological information measurement device according to the other modification is worn on a finger.

FIG. 14 schematically shows the structure of a biological information measurement device according to yet another modification.

### DESCRIPTION OF EMBODIMENT

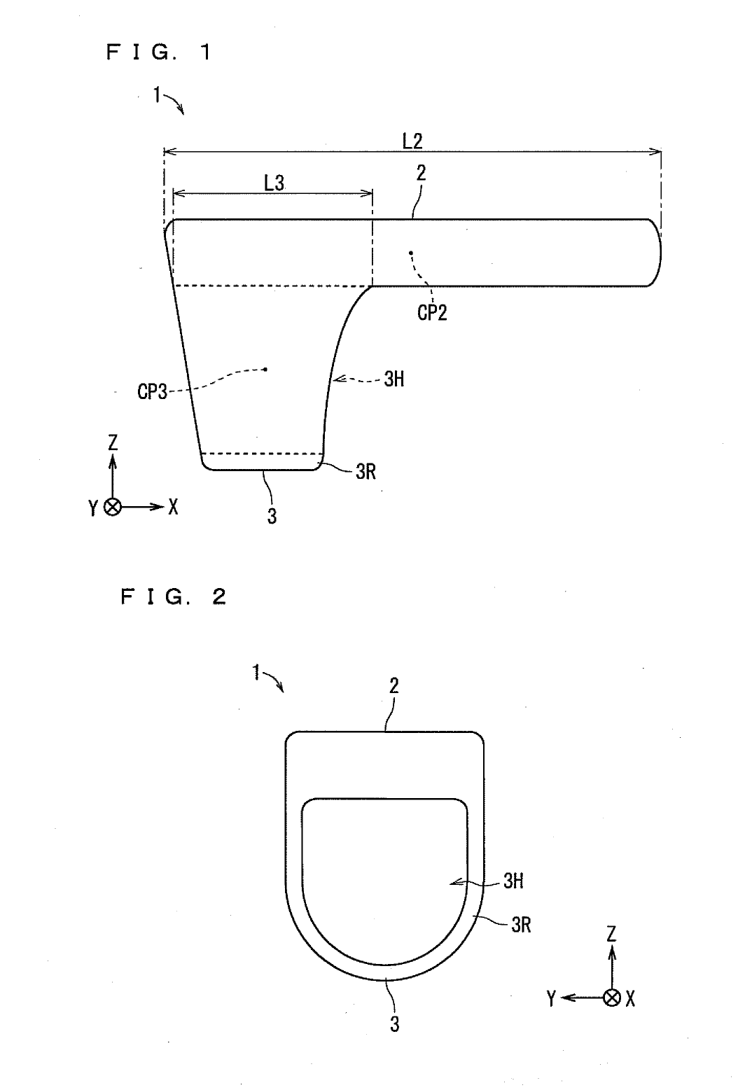

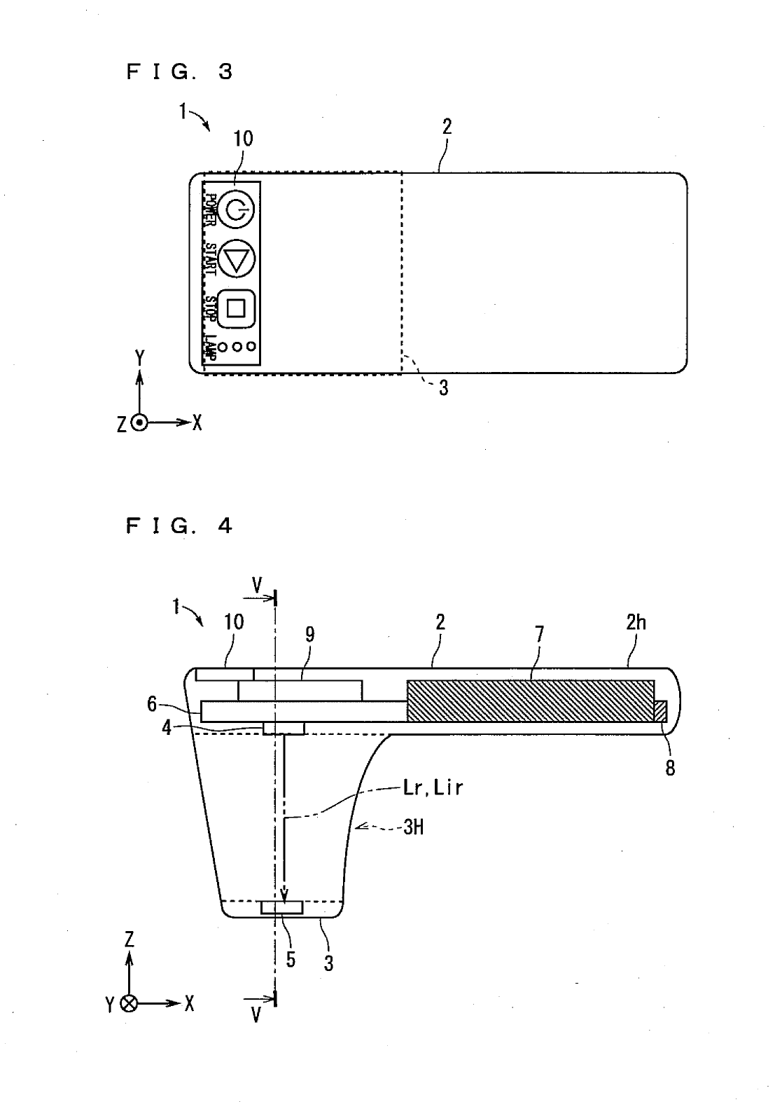

The following describes an embodiment of the present invention based on the drawings. It should be noted that components having a similar structure and function bear the same reference sign in the drawings, and repetition of description thereof is avoided below. The drawings are those shown schematically, and sizes of and positional relationships among various components in each of the drawings are not accurate. To each of FIGS. 1-5 and 8-12, a right-handed XYZ coordinate system in which one direction along a longitudinal direction of a biological information measurement device 1 (to the right, facing FIG. 1) is defined as a +X direction is assigned.

### (1) Embodiment

### (1-1) Structure of Biological Information Measurement Device

The biological information measurement device 1 according to the embodiment is, for example, a pulse oximeter that acquires a digital value concerning oxygen saturation of blood (an SpO₂ value) from a signal output from a light receiving unit 5 by the light receiving unit 5 receiving light that is emitted from a light source unit 4 and passes through a finger.

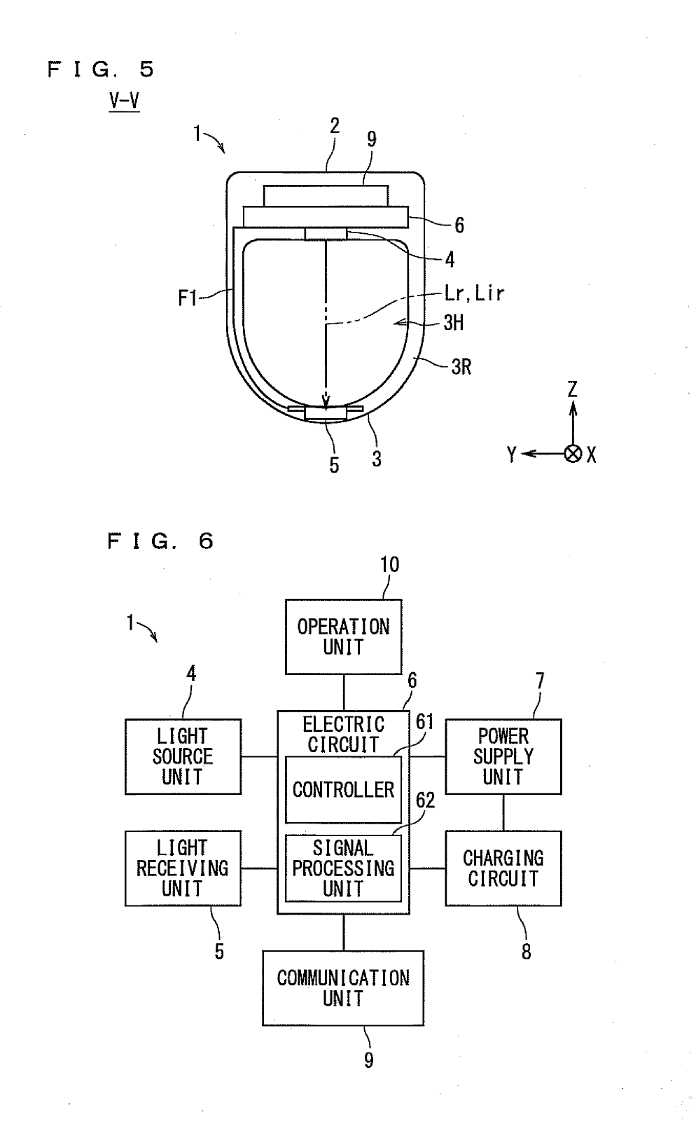

FIGS. 1-3 schematically show the appearance of the biological information measurement device 1. FIGS. 1, 2, and 3 are respectively a side view, a front view, and a top view of the biological information measurement device 1. FIGS. 4 and 5 schematically show the structure of the biological information measurement device 1. FIG. 5 shows a YZ cross section taken along an alternate long and short dash line V-V of FIG. 4.

As illustrated in FIGS. 1-3, the biological information measurement device 1 includes a body part 2 and a wearable part 3.

The body part 2 includes a housing 2h as well as an electric circuit 6, a power supply unit 7, a charging circuit 8, a communication unit 9, and an operation unit 10 that are arranged in the housing 2h. The housing 2h is approximately in a shape of a cuboid, for example. The housing 2h is harder than the wearable part 3 and has sufficient rigidity to make various components stored in the body part 2 less prone to breakage.

The wearable part 3 is fixed on the body part 2 and is to hold a finger of a living body when a variety of information on the living body is measured. When the wearable part 3 is softer than the housing 2h, the biological information measurement device 1 can provide a comfortable fit for subjects. The wearable part 3 preferably includes an elastic body that has elasticity to hold a finger, for example. Examples of the elastic body are a spring and a polymeric material such as rubber. Specifically, almost the entirety of the wearable part 3 may be made of resin, such as rubber, that has elasticity, and an approximately U-shaped leaf spring may be embedded in resin.

A length (also referred to as a first length) L2 of the body part 2 in an X direction, which is a longitudinal direction of the body part 2, is herein greater than a length (also referred to as a second length) L3 of the wearable part 3 in the X direction, which is the longitudinal direction. Since the body part 2 includes the electric circuit 6, the power supply unit 7, the charging circuit 8, the communication unit 9, and the operation unit 10 that are stored in the housing 2h as described above, the housing 2h is made rigid. In terms of not interfering with movement of a finger, shaping the body part 2 such that the body part 2 has a certain length along a longitudinal direction of the finger and providing the above-mentioned various components in the housing 2h are effective. For example, the first length L2 may be at least twice the second length L3. Furthermore, the center position CP3 of the wearable part 3 in the X direction is offset, from the center position CP2 of the body part 2 in the X direction, in one direction (−X direction) toward one end portion (one end portion in a −X direction) of the body part 2 in the longitudinal direction. The biological information measurement device 1 includes the light source unit 4 and the light receiving unit 5. The light source unit 4 and the light receiving unit 5 are arranged so as to oppose each other with the finger therebetween when the finger is held by the wearable part 3. The light source unit 4 and the light receiving unit 5 may be provided either in the body part 2 or in the wearable part 3.

When the wearable part 3 herein includes a ring part 3R that has an insertion hole 3H into which a finger of a living body is inserted in the −X direction, for example, the insertion hole 3H serves as an area in which the finger is held by the wearable part 3. In this case, the biological information measurement device 1 is worn on a finger quite easily by insertion of the finger into the insertion hole 3H. Furthermore, a burden imposed on the finger inserted into the insertion hole 3H is small, and thus a burden imposed on subjects by long-time wearing of the biological information measurement device 1 on the finger can further be reduced. When the ring part 3R is deformed by elasticity of the elastic body included in the wearable part 3 in a direction in which the insertion hole 3H closes, the biological information measurement device 1 can be stably worn on the finger.

The biological information measurement device 1 having the structure as described above is easily worn on a finger. Furthermore, in a state in which the biological information measurement device 1 is worn on the finger, movement to bend the finger at its joint is less likely to be interfered with by the biological information measurement device 1 as the wearable part 3 is small, and the body part 2 is less likely to protrude toward a fingertip. As a result, a burden imposed on subjects by long-time wearing of the device on the finger can be reduced.

### (1-2) Functional Structure of Biological Information Measurement Device

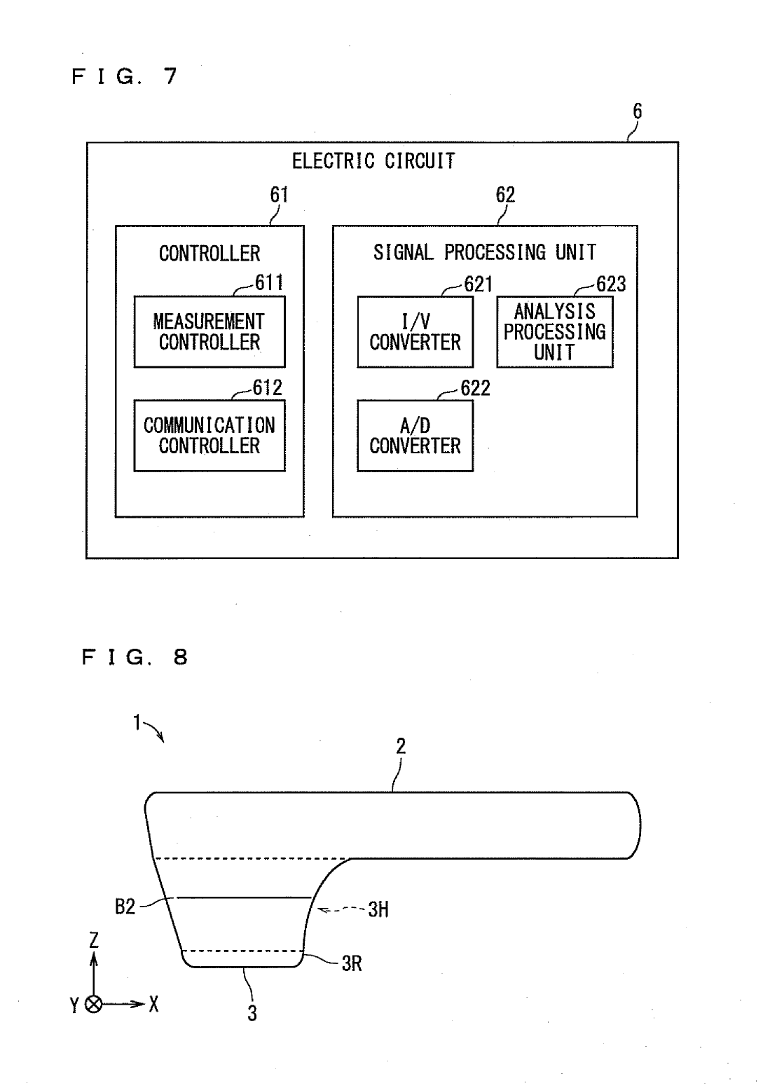

FIGS. 6 and 7 are block diagrams showing the functional structure of the biological information measurement device 1.

As shown in FIG. 6, the biological information measurement device 1 includes the light source unit 4, the light receiving unit 5, the electric circuit 6, the power supply unit 7, the charging circuit 8, the communication unit 9, and the operation unit 10.

The light source unit 4 emits light to the light receiving unit 5 by power supply from the power supply unit 7 in accordance with control performed by the electric circuit 6. In FIG. 5, a path along which the light travels (a light path) is indicated by an alternate long and two short dashes line. The light source unit 4 includes a part that emits light at a wavelength λ1 of red light and a part that emits light at a wavelength λ2 of infrared light. An example of the light source unit 4 is a light emitting diode (LED). The light source unit 4 alternately emits red light Lr at the wavelength λ1 and infrared light Lir at the wavelength λ2 during measurement.

The light receiving unit 5 outputs a current signal having a magnitude determined by intensity of received light to a signal processing unit 62, which is described later. The light receiving unit 5 includes a photoelectric conversion element, such as a silicon photodiode, that is at least sensitive to light at the wavelength λ1 and light at the wavelength λ2, for example. In a state in which a finger is inserted into the insertion hole 3H, the light receiving unit 5 receives, from among light at the wavelength λ1 and light at the wavelength λ2 that are emitted from the light source unit 4, light that passes through biological tissues of the finger. The light receiving unit 5 is electrically connected to the electric circuit 6 by wiring. The light receiving unit 5 may be provided in a flexible printed circuit (FPC: Flexible Printed Circuits) F1 that are electrically connected to the electric circuit 6, for example. With this structure, the current signal output from the light receiving unit 5 is transmitted to the electric circuit 6.

During measurement, the light source unit 4 alternately emits the red light Lr at the wavelength λ1 and the infrared light Lir at the wavelength λ2, and the light receiving unit 5 performs a light receiving operation in synchronization with the light emitting operation performed by the light source unit 4. The light emitting operation performed by the light source unit 4 and the light receiving operation performed by the light receiving unit 5 can be controlled by a controller 61, which is described later. An operation of projecting and receiving each of the light Lr and the light Lir is repeated in a cycle of approximately 1/100 seconds or more and 1/30 seconds or less, for example.

When the light source unit 4 is provided in the body part 2 or in a portion of the wearable part 3 that is closer to the body part 2, and the light receiving unit 5 is provided in the wearable part 3, a wiring path for supplying power to the light source unit 4 can be reduced. As a result, the effects of noise caused on the electric circuit 6 and other units by power supply to the light source unit 4 can be reduced.

The electric circuit 6 includes the controller 61 and the signal processing unit 62. The electric circuit 6 is preferably configured by various electronic components, integrated circuit components, a CPU, and the like. As shown in FIG. 7, the controller 61 includes a measurement controller 611, a communication controller 612, and a charging circuit controller (not shown). The signal processing unit 62 includes a current/voltage converter (hereinafter, referred to as an I/V converter) 621, an analog/digital converter (hereinafter, referred to as an A/D converter) 622, and an analysis processing unit 623.

The measurement controller 611 controls operations of the light source unit 4 and the light receiving unit 5. The measurement controller 611 herein causes the light source unit 4 to alternately emit the red light Lr at the wavelength λ1 and the infrared light Lir at the wavelength 22 each in a cycle of 1/100 seconds, for example. The communication controller 612 controls data communication performed by the communication unit 9, which is described later.

The I/V converter 621 converts a current signal periodically output from the light receiving unit 5 into a voltage signal. The voltage signal is a signal concerning an analog pulse wave (also referred to as a pulse wave signal). The A/D converter 622 converts the analog pulse wave signal output from the I/V converter 621 into a digital pulse wave signal. As a result, a digital value concerning a pulse wave can be acquired.

The analysis processing unit 623 performs predetermined data analysis based on the digital pulse wave signal output from the A/D converter 622, thereby calculating various values such as a value of the amount of each of the light Lr and the light Lir received by the light receiving unit 5, a value of amplitude of a pulse wave of each of the light Lr and the light Lir, a value of a ratio of the amplitude of the red light Lr to the amplitude of the infrared light Lir, a value of oxygen saturation of blood (an SpO₂ value), a value of a pulse rate, a value of a pulse wave interval (cycle).

The measurement controller 611, the communication controller 612, and the analysis processing unit 623 may be configured by a dedicated electronic circuit, or may be achieved by a microprocessor, a digital signal processor (DSP), and the like executing a program.

The power supply unit 7 includes a secondary battery, such as a nickel hydrogen battery or a lithium ion battery. The power supply unit 7 supplies power to various components of the biological information measurement device 1, such as the electric circuit 6 and the light source unit 4. This eliminates the need for a mechanism for replacing a primary battery, such as a dry battery, in the body part 2. As a result, the body part 2 can have a simple and durable structure.

The charging circuit 8 is a circuit for charging the secondary battery included in the power supply unit 7. For example, the secondary battery is charged by connecting a charger to a terminal that is electrically connected to the secondary battery. As a result, the secondary battery can be charged with a simple configuration. When the charging circuit 8 charges the secondary battery without contact, i.e., when the charging circuit 8 includes a circuit for charging the second battery without contact, for example, a terminal and the like for connecting the charger and the like are unnecessary. As a result, the secondary battery can be charged with a simpler configuration. As a method for performing charging without contact, a method of making use of electromagnetic induction in a coil and the like can be used.

The communication unit 9 wirelessly transmits data acquired by the signal processing unit 62. With this structure, a component for analyzing and storing a signal and a display unit for displaying measurement results can be omitted. As a result, reduction in size of the device, power saving, and reduction in manufacturing cost can be achieved.

The communication unit 9 may transmit the digital pulse wave signal acquired by the A/D converter 622 included in the signal processing unit 62, i.e., data of a digital value concerning a pulse wave, for example. In this case, it is sufficient that an external device (e.g., a personal computer) that has received data transmitted from the communication unit 9 calculates various values by using a component corresponding to the analysis processing unit 623. As a result, the structure for processing a signal in the biological information measurement device 1 can be simplified. Therefore, reduction in size of the device, power saving, and reduction in manufacturing cost can further be achieved.

Assume herein that the signal processing unit 62 acquires, based on the digital pulse wave signal, a digital value concerning one or more of a value of oxygen saturation of blood (an SpO₂ value), a pulse rate, and a pulse wave interval (cycle). In this case, the communication unit 9 can transmit data of the digital value concerning one or more of the value of oxygen saturation of blood (an SpO₂ value), the pulse rate, and the pulse wave interval (cycle) acquired by the signal processing unit 62. As a result, an external device that has received the data transmitted from the communication unit 9 can easily acquire useful information without performing any special arithmetic operation. Furthermore, since a display unit for displaying measurement results in the biological information measurement device 1 can be omitted, reduction in size of the device, power saving, and reduction in manufacturing cost can be achieved.

The electric circuit 6 may include a variety of memory for storing data acquired by the signal processing unit 62.

The operation unit 10 includes a power button, a measurement start button, and a measurement termination button, for example. The power button is a button for performing switching between supply and no supply of power from the power supply unit 7 to each component of the biological information measurement device 1. The measurement start button is a button for starting measurement of a value of oxygen saturation of blood (an SpO₂ value) or the like. The measurement termination button is a button for terminating measurement of a value of oxygen saturation of blood (an SpO₂ value) or the like.

### (1-3) Wearing of Biological Information Measurement Device on Finger

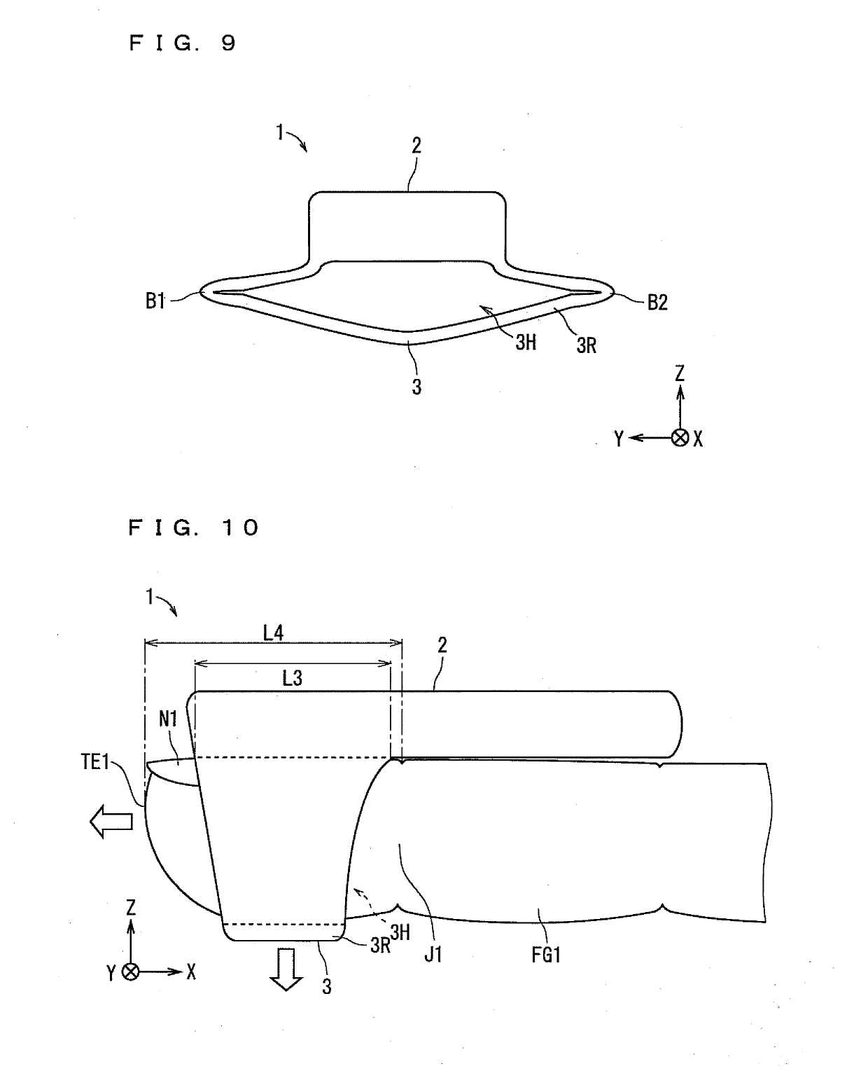

Ms, 8-10 schematically show one example of how the biological information measurement device 1 is worn on a finger FG1. FIGS. 8 and 9 illustrate one form of the biological information measurement device 1 in a state in which the finger FG1 is not inserted into the insertion hole 3H. FIG. 10 illustrates one form of the biological information measurement device 1 in a state in which the finger FG1 is inserted into the insertion hole 3H. The finger FG1 is herein held by the wearable part 3 in a state in which the finger FG1 extends along the X direction, which is the longitudinal direction of the body part 2. In this case, the longitudinal direction of the body part 2 may not completely coincide with the direction along which the finger FG1 extends. For example, the finger FG1 may be slightly inclined relative to the body part 2 in a direction of rotation around an imaginary axis that is approximately parallel to the Z axis. The following describes one example of how the biological information measurement device 1 is worn on the finger FG1.

For example, as illustrated in FIGS. 8 and 9, in a state in which the biological information measurement device 1 is not worn on a finger, the elastic body included in the wearable part 3 demonstrates elasticity to hold a finger inserted into the insertion hole 3H, and the ring part 3R is elastically deformed in the Z direction, which is a direction in which the insertion hole 3H closes. In this case, as illustrated in FIG. 9, the ring part 3R can be bent at positions B1 and B2 located on ±Y side thereof, for example.

When the biological information measurement device 1 is worn on a finger, the ring part 3R is elastically deformed such that the insertion hole 3H is expanded in a −Z direction against elasticity of the elastic body included in the wearable part 3, and, as illustrated in FIG. 10, the finger FG1 is inserted into the insertion hole 3H in the −X direction. As a result, in a state in which the biological information measurement device 1 is worn on the finger FG1, elasticity of the elastic body included in the wearable part 3 attempts to deform the ring part 3R in the Z direction, which is a direction in which the insertion hole 3H closes, and thus the finger FG1 is held by the wearable part 3. In this case, it is sufficient that the finger FG1 is inserted into the insertion hole 3H so that the light source unit 4 shines the light Lr and the light Lir onto an area of the finger FG1 inserted into the insertion hole 3H between a nail N1 and a distal interphalangeal joint (also referred to as a first joint) J1.

As illustrated in FIG. 10, it is sufficient that the second length L3 of the wearable part 3 in the X direction is smaller than a length (also referred to as a third length) L4 from a tip end portion TE1 to the first joint J1 of the finger FG1 in a longitudinal direction of the finger FG1 (herein, the X direction) In this case, when the body part 2 is placed on a side of the nail N1 (a back side) of the finger FG1, movement to bend the finger FG1 at the first joint J1 is less likely to be interfered with by both of the body part 2 and the wearable part 3. As a result, the biological information measurement device 1 is easily worn on a fingertip, and a burden imposed on subjects by long-time wearing of the biological information measurement device 1 on the fingertip can be reduced.

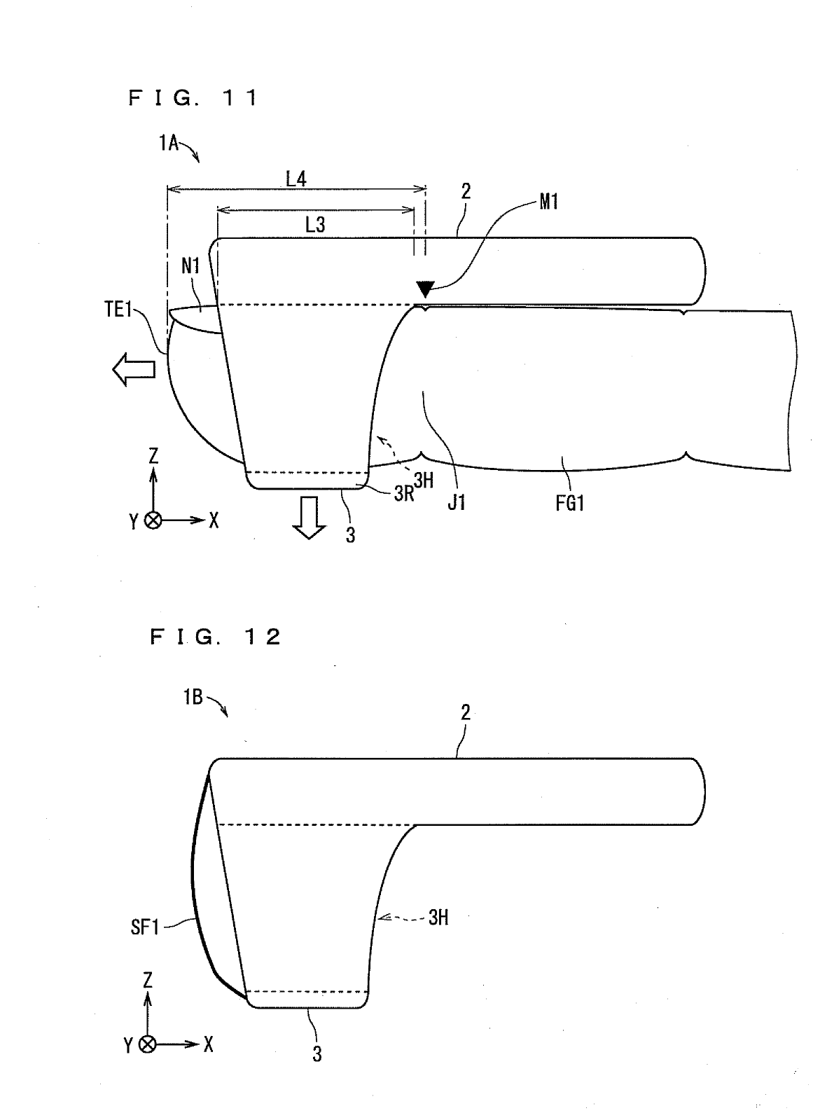

At least one of the body part 2 and the wearable part 3 may be provided with a mark that indicates a position, in the longitudinal direction of the body part 2, at which the finger FG1 is to be placed when the finger FG1 is held by the wearable part 3. For example, as illustrated in FIG. 11, a biological information measurement device 1A that is produced based on the above-mentioned biological information measurement device 1, and includes the body part 2 that is provided with a mark M1 that indicates the position at which the first joint J1 of the finger FG1 should be placed may be used. By virtue of the mark M1 as described above, the biological information measurement device 1A can quickly be worn on the finger FG1 at a position onto which light is to be shined. That is to say, positioning of the biological information measurement device 1A is easy in wearing the biological information measurement device 1A on the finger FG1. Furthermore, since the biological information measurement device 1A is worn on the finger FG1 at an appropriate position, the finger FG1 can be moved freely at the first joint J1, leading to reduction of a burden on the finger FG1.

### (1-4) Summary of Embodiment

As described above, in the biological information measurement device 1 according to the embodiment, the light source unit 4 and the light receiving unit 5 are arranged so as to oppose each other with a finger therebetween when the finger is held by the wearable part 3. In addition, the first length L2 of the body part 2 in the longitudinal direction of the body part 2 is greater than the second length L3 of the wearable part 3 in the longitudinal direction, and the center position CP3 of the wearable part 3 is offset, from the center position CP2 of the body part 2, toward one end portion of the body part 2 in the longitudinal direction. As a result, the biological information measurement device 1 can easily be worn on the finger. In a state in which the biological information measurement device 1 is worn on the finger, movement to bend the finger at its joint is less likely to be interfered with by the biological information measurement device 1 as the wearable part 3 is small, and the body part 2 is less likely to protrude toward a fingertip. A burden imposed on subjects by long-time wearing of the biological information measurement device 1 on the finger can be reduced.

### (2) Modifications

It should be noted that the present invention is in no way limited to the aforementioned embodiment, and can be implemented by making various modifications and improvements without departing from the scope of the present invention.

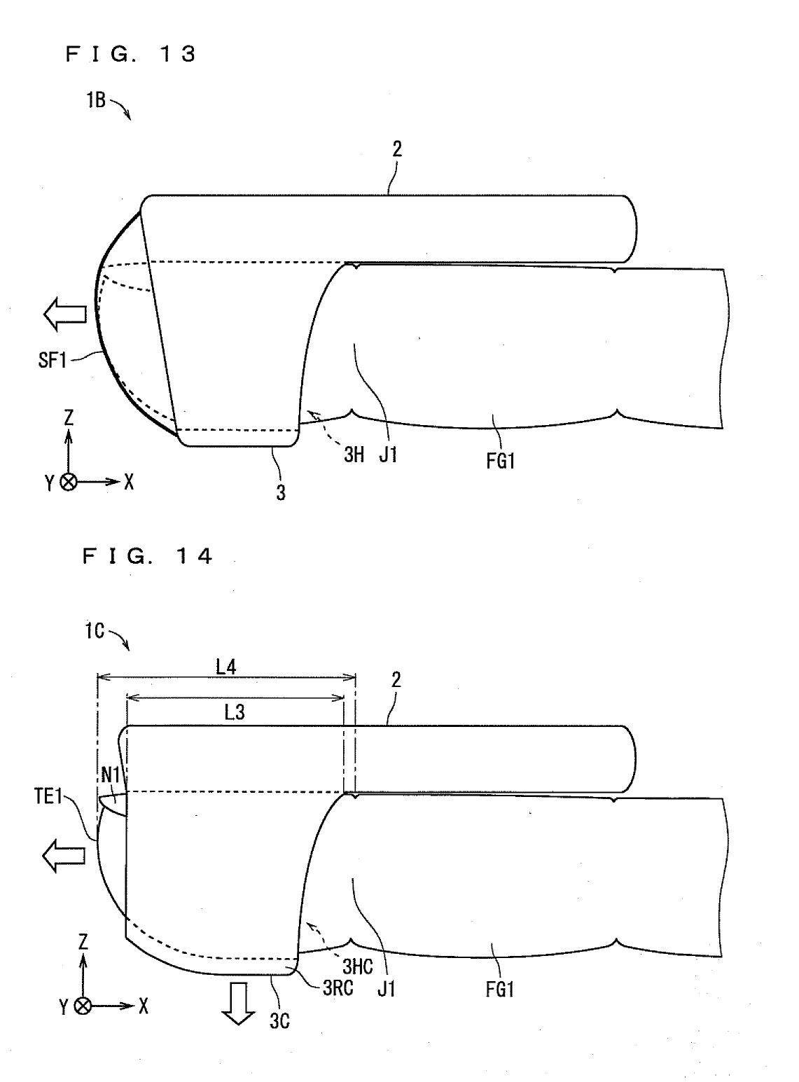

For example, although the insertion hole 3H is a though-hole provided in the ±X direction in the biological information measurement device 1 according to the aforementioned embodiment, the biological information measurement device 1 may not have this structure. For example, as illustrated in FIG. 12, a biological information measurement device 1B that is obtained by adding, to the biological information measurement device 1, a stopper part SF1 at one end portion of the insertion hole 3H of the wearable part 3 in the −X direction may be used. In this case, as illustrated in FIG. 13, by inserting the finger FG1 into the insertion hole 3H in the +X direction, and sufficiently bringing the finger FG1 into contact with the stopper part SF1, the biological information measurement device 1A can be quickly and appropriately worn on the finger FG1 at the position onto which light is to be shined. That is to say, positioning of the biological information measurement device 1A is easy in wearing the biological information measurement device 1A on the finger FG1. Furthermore, since the biological information measurement device 1B is worn on the finger FG1 at an appropriate position, the finger FG1 can be moved freely at the first joint J1, leading to reduction of a burden on the finger FG1.

When the stopper part SF1 is a member that has elasticity, such as rubber, the biological information measurement device 1A can easily be worn on a fingertip as the fingertip is less likely to be damaged when the finger FG1 is inserted into the insertion hole 3H. Furthermore, the fingertip easily fits the stopper part SF1, and thus a burden imposed on subjects by long-time wearing of the biological information measurement device 1A on the fingertip can be reduced. When the stopper part SF1 is a light blocking part that blocks passage of light, outside light is less likely to be shined onto the light receiving unit 5 due to the presence of the light blocking part. As a result, the effects of noise and measurement errors are less likely to be caused.

As illustrated in FIG. 14, a biological information measurement device 1C that includes, in place of the insertion hole 3H, an insertion hole 3HC whose diameter decreases with decreasing distance from one end portion of the insertion hole 3HC in the X direction may be used. With the above-mentioned change, the wearable part 3 and the ring part 3R are respectively changed to a wearable part 3C and a ring part 3RC. In this case, the biological information measurement device 1C can be quickly and appropriately worn on the finger FG1 at the position onto which light is to be shined by inserting the finger FG1 into the insertion hole 3HC from the +X side thereof, and sufficiently bringing the finger FG1 into contact with a portion of the ring part 3R in which the diameter of the insertion hole 3HC decreases. That is to say, positioning of the biological information measurement device 1C is easy in wearing the biological information measurement device 1C on the finger FG1 as in the above-mentioned case where the stopper part SF1 is provided.

Although the light source unit 4 is provided in the body part 2 or in a portion of the wearable part 3 that is closer to the body part 2, and the light receiving unit 5 is provided in the wearable part 3 in the biological information measurement device 1 according to the above-mentioned embodiment, the biological information measurement device 1 may not have this structure. For example, the light source unit 4 may be provided in the wearable part 3, and the light receiving unit 5 may be provided in the body part 2 or in a portion of the wearable part 3 that is closer to the body part 2. Alternatively, both of the light source unit 4 and the light receiving unit 5 may be provided in the wearable part 3, for example. In terms of reducing the effects of noise caused on the electric circuit 6 and other units by power supply to the light source unit 4, however, it is preferable to provide the light source unit 4 in the body part 2 or in a portion of the wearable part 3 that is closer to the body part 2, and to provide the light receiving unit 5 in the wearable part 3.

Although a display unit is not provided in the biological information measurement device 1 according to the above-mentioned embodiment, the biological information measurement device 1 may not have this structure. For example, a display unit for displaying various values acquired by the analysis processing unit 623 may be provided. In this case, the communication controller 612 and the communication unit 9 may be omitted.

Although the wearable part 3 is fixed to one end portion of the body part 2 in the biological information measurement device 1 according to the above-mentioned embodiment, the biological information measurement device 1 may not have this structure. For example, the body part 2 may have a portion that slightly protrudes in the −X direction further than the wearable part 3. That is to say, it is sufficient that the center position CP3 of the wearable part 3 is offset, from the center position CP2 of the body part 2, toward one end portion of the body part 2 in the longitudinal direction.

Although the wearable part 3 includes the ring part 3R into which the finger FG1 is inserted in the biological information measurement device 1 according to the above-mentioned embodiment, the biological information measurement device 1 may not have this structure. For example, the wearable part 3 may have a clip structure that holds the finger FG1. In terms of reducing a burden imposed on subjects by long-time wearing on the finger, however, it is desirable that the wearable part 3 include the ring part 3R into which the finger FG1 is inserted. It is more desirable that the ring part 3R be deformed by elasticity of the wearable part 3 in a direction in which the insertion hole 3H closes.

Although the signal processing unit 62 acquires a digital value concerning oxygen saturation of blood (an SpO₂ value) in the biological information measurement device 1 according to the above-mentioned embodiment, the biological information measurement device 1 may not have this structure. For example, a biological information measurement device, other than a pulse oximeter, that measures biological information concerning a pulse wave, such as a heart rate, without acquiring the SpO₂ value may be used.

It should be appreciated that all or part of the embodiment and various modifications set forth above can appropriately be combined with one another within a reasonable scope.

### REFERENCE SIGNS LIST

1, 1A, 1B, 1C Biological information measurement device
2 Body part
2h Housing
3, 3C Wearable part
3H, 3HC Insertion hole
3R, 3RC Ring part
4 Light source unit
5 Light receiving unit
6 Electric circuit
7 Power supply unit
8 Charging circuit
9 Communication unit
61 Controller
62 Signal processing unit
621 Current/voltage converter (I/V converter)
622 Analog/digital converter (A/D converter)
623 Analysis processing unit
CP2, CP3 Center position
J1 Distal interphalangeal joint (first joint)
SF1 Stopper part
TE1 Tip end portion

1. A biological information measurement device comprising:
a body part; and
a wearable part that is fixed on the body part and is to hold a finger of a living body, wherein
the biological information measurement device includes a light source unit and a light receiving unit that are arranged so as to oppose each other with said finger therebetween when said finger is held by said wearable part,
said body part includes an electric circuit and a power supply unit, the electric circuit including a signal processing unit,
said signal processing unit acquires a digital value concerning a pulse wave from a signal output from said light receiving unit, the signal being output by said light receiving unit receiving light that is emitted from said light source unit and passes through said finger, and
a first length of said body part in a longitudinal direction of the body part is greater than a second length of said wearable part in the longitudinal direction.

2. The biological information measurement device according to claim 1, wherein
said wearable part is provided so as to hold said finger in a state in which said finger extends along the longitudinal direction of said body part.

3. The biological information measurement device according to claim 1, wherein
a center position of said wearable part in said longitudinal direction is offset, from a center position of said body part in said longitudinal direction, in one direction toward one end portion of said body part in said longitudinal direction.

4. The biological information measurement device according to claim 1, wherein
said first length is at least twice said second length.

5. The biological information measurement device according to claim 1, wherein
said second length is smaller than a third length from a tip end portion to a distal interphalangeal joint of said finger.

6. The biological information measurement device according to claim 5, wherein
a mark that indicates a position, in the longitudinal direction of said body part, at which said finger is to be placed when said finger is held by said wearable part is provided.

7. The biological information measurement device according to claim 1, wherein
said wearable part includes an elastic body that has elasticity to hold said finger.

8. The biological information measurement device according to claim 1, wherein
said wearable part includes a ring part that has an insertion hole into which said finger is inserted in one direction toward one end portion of said body part in said longitudinal direction.

9. The biological information measurement device according to claim 8, wherein
said ring part is deformed by elasticity in a direction in which said insertion hole closes.

10. The biological information measurement device according to claim 8, wherein
said wearable part includes a stopper part that is provided at one end portion of said insertion hole in said one direction.

11. The biological information measurement device according to claim 10, wherein
said stopper part includes a light blocking part.

12. The biological information measurement device according to claim 8, wherein
a diameter of said insertion hole of said wearable part decreases with decreasing distance from one end portion of said insertion hole in said one direction.

13. The biological information measurement device according to claim 1, wherein
said light source unit is provided in said body part or in a portion of said wearable part that is closer to said body part, and
said light receiving unit is provided in said wearable part.

14. The biological information measurement device according to claim 1, wherein
said body part includes a housing that is harder than said wearable part.

15. The biological information measurement device according to claim 1, wherein
said power supply unit includes a secondary battery.

16. The biological information measurement device according to claim 15, wherein
said body part further includes a charging circuit that charges said secondary battery.

17. The biological information measurement device according to claim 16, wherein
said charging circuit includes a circuit that charges said secondary battery without contact.

18. The biological information measurement device according to claim 1, wherein
said body part further includes a communication unit that wirelessly transmits data acquired by said signal processing unit.

19. The biological information measurement device according to claim 18, wherein
said communication unit transmits data of the digital value concerning said pulse wave acquired by said signal processing unit.

20. The biological information measurement device according to claim 18, wherein
said signal processing unit acquires, based on the digital value concerning said pulse wave, a digital value concerning one or more of oxygen saturation of blood, a pulse rate, and a pulse wave interval, and
said communication unit transmits data of the digital value concerning one or more of said oxygen saturation of blood, said pulse rate, and said pulse wave interval acquired by said signal processing unit.

21. A pulse oximeter comprising:
a body part; and
a wearable part that is fixed on the body part and is to hold a finger of a living body, wherein
the pulse oximeter includes a light source unit and a light receiving unit that are arranged so as to oppose each other with said finger therebetween when said finger is held by said wearable part,
said body part includes an electric circuit and a power supply unit, the electric circuit including a signal processing unit,
said signal processing unit acquires a value concerning oxygen saturation of blood from a signal output from said light receiving unit, the signal being output by said light receiving unit receiving light that is emitted from said light source unit and passes through said finger, and
a first length of said body part in a longitudinal direction of the body part is greater than a second length of said wearable part in the longitudinal direction.

22. The pulse oximeter according to claim 21, wherein
said wearable part is provided so as to hold said finger in a state in which said finger extends along the longitudinal direction of said body part.
